(19) United States
(12) Patent Application Publication
(10) Pub. No.: US 2023/0101706 A1
(43) Pub. Date: Mar. 30, 2023

(54) DUAL-FUNCTION ANCHOR SYSTEM

(71) Applicant: Trace Orthopedics, LLC, Philadelphia, PA (US)

(72) Inventors: William B. Morrison, Delran, NJ (US); Adam J. Greenspan, Voorhees, NJ (US)

(21) Appl. No.: 18/061,332

(22) Filed: Dec. 2, 2022

Related U.S. Application Data
(63) Continuation of application No. PCT/US2021/035838, filed on Jun. 4, 2021.
(60) Provisional application No. 63/034,895, filed on Jun. 4, 2020.

(51) Int. Cl. A61F 2/08

(52) U.S. Cl. CPC A61F 2/0811; A61F 2002/0858

(57) ABSTRACT

The present invention provides a surgical anchor comprising a screw and a coil; said coil having a conical shape, wound around the screw shaft with the first end of the coil being tapered and having a ring circumference smaller than that of the screw head and being engaged to the bottom of the screw head and the second end of the coil being flared with a larger ring circumference and positioned along the screw shaft.

### CROSS-REFERENCE TO RELATED APPLICATIONS

This application is a continuation under 35 U.S.C. § 120 of International Application No. PCT/US2021/035838, filed Jun. 4, 2021, which claims the benefit of priority to U.S. Provisional Patent Application No. 63/034,895, filed Jun. 4, 2020, the contents of which are incorporated by reference herein in their entirety.

### BACKGROUND OF INVENTION

Movement of the human body requires complex communication between the structural components, namely the muscles, tendons, bones, and vasculature, and the electrical pulses that control the muscles to create movement. Over time, the body ages, namely the muscles, tendons, and bones wear and damage can occur. A relatively commonplace injury involves complete or partial separation of ligaments, tendons or other soft tissues from their associated human bones. Complete or partial separation of the ligaments, tendons and other soft tissue is relatively common place in athletes and typically result from excess stress being placed upon such tissue. Of course, soft tissue separation or detachment from human bone may likewise occur as a result of an accident such as a fall, over exertion during a work related activity, during the course of physical activity, or in several different situations involving human activity.

In many cases, injuries of partial detachment fail to heal naturally, causing the patient chronic pain and/or ongoing discomfort despite methods to treat using conservative management techniques and procedures. Such injuries are typically repaired with open surgery, as it is otherwise difficult to properly secure the tissue and ensure recovery. Accordingly, a number of surgical procedures have been devised for reattaching such detached or separated tissue. Moreover, surgical techniques have advanced such that severely damaged ligaments and/or tendons can now be replaced through surgery.

One such technique involves reattachment of the detached or separated tissue using “traditional” attachment devices such as metal staples, sutures over buttons, and cancellous bone screws. These “traditional” devices have also been used in connection with the attachment of ligaments or tendons that have been harvested from other parts of the human body and are being used to replace or repair severely damaged tissues. It should be appreciated, however, that “traditional” repair methods have not been uniformly successful. As an example, rigid attachment of ligaments and tendons using “traditional” attachment devices such as staples, screws and sutures cannot be maintained when extreme tensile loads are applied thereto.

Given the risks of comorbidity and infection as well as the possibility of “traditional” device failure, it is of tremendous interest and public health benefit to develop a novel device with the ability to achieve functional re-attachment of soft tissues to bone using a minimally invasive approach following the principles of conservative management. To optimally heal this type of injury in a minimally invasive fashion, a specialized system is needed which includes components used in vivo and ex vivo. Biocompatibility and mechanical performance are critical aspects of the implantable components in such a system, and a given implantation site carries distinct considerations relating to both biocompatibility and mechanical performance.

For anchoring within bone tissue, rigid-high strength material is needed to effectively position the device into bone tissue. This includes generating sufficient torque to penetrate the bone tissue as well as possessing sufficient strength to establish and maintain fixation for the desired time period, in this case at least 6 months. For securing soft tissues, a ductile material is needed to effectively position the device into soft tissue for guidance into the bone anchor. This includes deforming temporarily under force exerted associated with positioning while maintaining appropriate strength. The ability to accomplish these functions is determined by the component composition. Parameters such as elastic modulus, tensile strength, shear strength, flexural strength shall guide the selection of suitable materials for the respective components.

### SUMMARY OF THE INVENTION

In certain aspects, the present invention provides a surgical anchor comprising a screw and a coil; said coil having a conical shape, wound around the screw shaft with the first end of the coil being tapered and having a ring circumference smaller than that of the screw head and being engaged to the bottom of the screw head and the second end of the coil being flared with a larger ring circumference and positioned along the screw shaft.

In certain aspects, the present invention provides a surgical system comprising a needle comprising an aperture of sufficient diameter for receiving a surgical anchor comprising a screw and a coil; a stylet tool, insertable into said aperture for inserting said anchor and capable of engaging with and turning the screw; said screw comprising a shaft having threads and a head, said head having a bottom face, and said coil having a conical shape, wound around the screw shaft with the first end of the coil being tapered and having a ring circumference smaller than that of the screw head and being engaged to the bottom of the screw head and the second end of the coil being flared with a larger ring circumference and positioned along the screw shaft. In some embodiments, the screw or coil described herein includes one or more materials selected from: poly(L-lactic acid), poly(D-L-lactic acid), poly(lactic-co-glycolic acid), poly(p-dioxanone), poly(propylene fumarate), copolymers of poly(L-lactic acid and poly(lactic-co-glycolic acid), magnesium based alloys including Mg—Zn, Mg-6Zn, Mg—Zn—Ca, Mg—Ca—Sr, and MgYREZr, and iron-based alloys including Fe—Mn.

In some embodiments, the screw or coil described herein includes a polymer and/or a coating on a metal, wherein the polymer or coating comprises one or more materials of claim 9.

In some embodiments, the screw or coil described herein includes a ceramic material comprising calcium phosphate, tricalcium phosphate, and hydroxyapatite in a particulate-reinforced polymer matrix and a coating comprising a ceramic material comprising calcium phosphate, tricalcium phosphate, and hydroxyapatite.

In some embodiments, the screw described herein has shape-memory properties. In some embodiments, the screw or coil described herein is non-absorbable.

In certain aspects, the present invention provides a method of repairing a soft tissue when disassociated from a bone comprising: inserting a surgical anchor into a soft tissue which has become detached from bone, said system comprising an aperture for inserting surgical anchor comprising a screw and a coil, and a stylet tool insertable into said aperture for inserting said anchor and capable of engaging with and turning the screw to cause its entry into the bone; said screw comprising a shaft having threads, and a head, said head having a bottom face, and a coil; said coil having a conical shape, wound around the screw shaft with the first end of the coil being tapered and having a ring circumference smaller than that of the screw head and being engaged to the bottom of the screw head and the second end of the coil being flared with a larger ring circumference and positioned along the screw shaft.

In some aspects, the present invention provides a surgical anchor comprising a screw and a coil, said screw comprising a head, a shaft having threads, and a collar, said collar having a greater cross-sectional area than that of the shaft and the head. In some embodiments, the screw includes a collar having threads in the same direction as the threads of the screw shaft. In some embodiments, the anchor includes a coil having one or more selected from: a conical shape, wound around the screw shaft with the first end of the coil being tapered and having a ring circumference smaller than that of the screw head and being engaged to the collar and the second end of the coil being flared with a larger ring circumference and positioned along the screw shaft, and; a cylindrical shape, wound around the screw shaft with the first end of the coil engaged to the collar and the second end of the coil positioned along the screw shaft.

In certain aspects, the present invention provides a method of repairing a soft tissue when disassociated from a bone comprising: inserting a surgical anchor into a soft tissue which has become detached from bone, said system comprising an aperture for inserting surgical anchor comprising a screw and a coil, and a stylet tool insertable into said aperture for inserting said anchor and capable of engaging with and turning the screw to cause its entry into the bone; said screw comprising a head, a shaft having threads, and a collar, said collar having a greater cross-sectional area than that of the shaft and the head. In some embodiments, the screw includes a collar having threads in the same direction as the threads of the screw shaft. In some embodiments, the anchor includes coil having one or more selected from: a conical shape, wound around the screw shaft with the first end of the coil being tapered and having a ring circumference smaller than that of the screw head and being engaged to the collar and the second end of the coil being flared with a larger ring circumference and positioned along the screw shaft; and, a cylindrical shape, wound around the screw shaft with the first end of the coil engaged to the collar and the second end of the coil positioned along the screw shaft.

In some aspects, the present invention provides a surgical anchor comprising a screw and a coil, said screw comprising a head, said head having a bottom face, and a shaft having threads, said shaft comprising a section with a greater cross-sectional area than the rest of the shaft such that the shaft tapers from this section in both directions toward and away from the head.

In some embodiments, the anchor includes a coil having one or more selected from:

a conical shape, wound around the screw shaft with the first end of the coil being tapered and having a ring circumference smaller than that of the screw head and being engaged to the bottom face of the screw head, and the second end of the coil being flared with a larger ring circumference and positioned along the screw shaft; and, a cylindrical shape, wound around the screw shaft with the first end of the coil engaged to the bottom face of the screw head, and the second end of the coil positioned along the screw shaft.

In certain aspects, the present invention relates to a method of repairing a soft tissue when disassociated from a bone comprising: inserting a surgical anchor into a soft tissue which has become detached from bone, said system comprising an aperture for inserting surgical anchor comprising a screw and a coil, and a stylet tool insertable into said aperture for inserting said anchor and capable of engaging with and turning the screw to cause its entry into the bone; said screw comprising a head, said head having a bottom face, and a shaft having threads, said shaft comprising a section with a greater cross-sectional area than the rest of the shaft such that the shaft tapers from this section in both directions toward and away from the head, wherein the coil is positioned between the screw head and the section with a greater cross-sectional area.

In some embodiments the method includes a coil having one or more selected from:

a conical shape, wound around the screw shaft with the first end of the coil being tapered and having a ring circumference smaller than that of the screw head and being engaged to the collar and the second end of the coil being flared with a larger ring circumference and positioned along the screw shaft, a cylindrical shape, wound around the screw shaft with the first end of the coil engaged to the collar and the second end of the coil positioned along the screw shaft.

In certain aspects, the present invention relates to the use of a dual-action surgical system for securing soft tissue to a bone, said surgical system comprising an applicator having a needle and an aperture for inserting a surgical anchor, said surgical anchor comprising a screw comprising a shaft having threads and a coil; said coil wound around the screw shaft comprising a coil end which is defined to engage at least one surface of the screw and a second coil end; engaging the end of the shaft and the second coil end to a soft tissue.

In certain aspects, the present invention provides a surgical anchor having: a fastener; and an external invertible coil coupled to a proximal end of the fastener. In some embodiments, the fastener is a screw having: distal threads; and a reamer proximal to the distal threads, wherein the screw is a headless screw; and the screw comprises an annular recess proximal to the reamer, the recess adapted and configured to receive a portion of the external invertible coil. In some embodiments, the screw includes a head having an outer diameter equal to or less than an outer diameter of the reamer.

In certain aspects, the present invention provides a surgical anchor including: a screw comprising: distal threads; and a proximal head; and a coil coupled to the screw proximal to the distal threads; wherein: the screw can rotate freely within the coil in either rotational direction without driving the coil; or the diameter of the coil expands distally.

In certain aspects, the present invention provides a method of repairing a soft tissue when disassociated from a bone, the method comprising: driving the surgical anchor described herein through the soft tissue and into the bone until at least a portion of the coil lies against the soft tissue proximal to the head of the screw

### BRIEF DESCRIPTION OF THE DRAWINGS

FIG. 1 depicts a needle inserted adjacent to soft tissue, which is separated from the bone.

FIG. 2 depicts the needle inserted into soft tissue.

FIGS. 3A-3C depict details of an embodiment of a screw with a wound conical coil.

FIG. 3B depicts the top view of a screw with a wound conical coil showing concentric rings.

FIG. 4C depicts a screw with a wound conical coil that is inverted.

FIGS. 4A and 4B depict a screw with a wound conical coil being deployed through a needle into soft tissue. FIG. 6A depicts use of a screwdriver through the interior of the needle;

FIG. 6B depicts the top face of the screw head comprising an inlet for the screwdriver, here an inverted pyramid shape.

FIG. 5 depicts advancing the screw into the bone to secure the soft tissue to the bone.

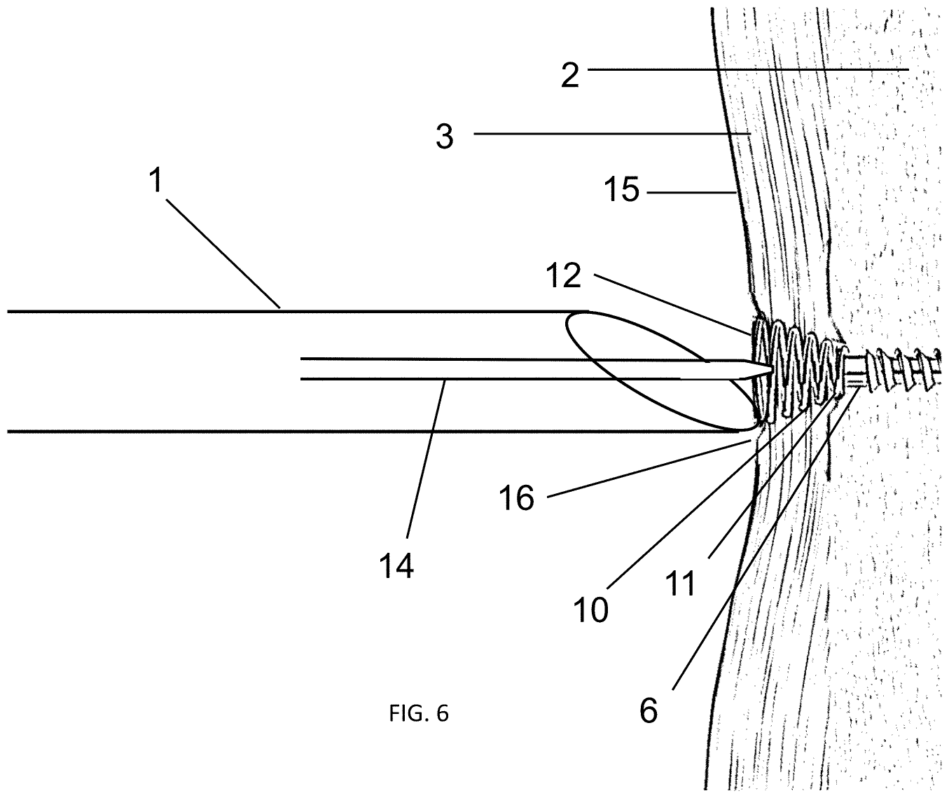

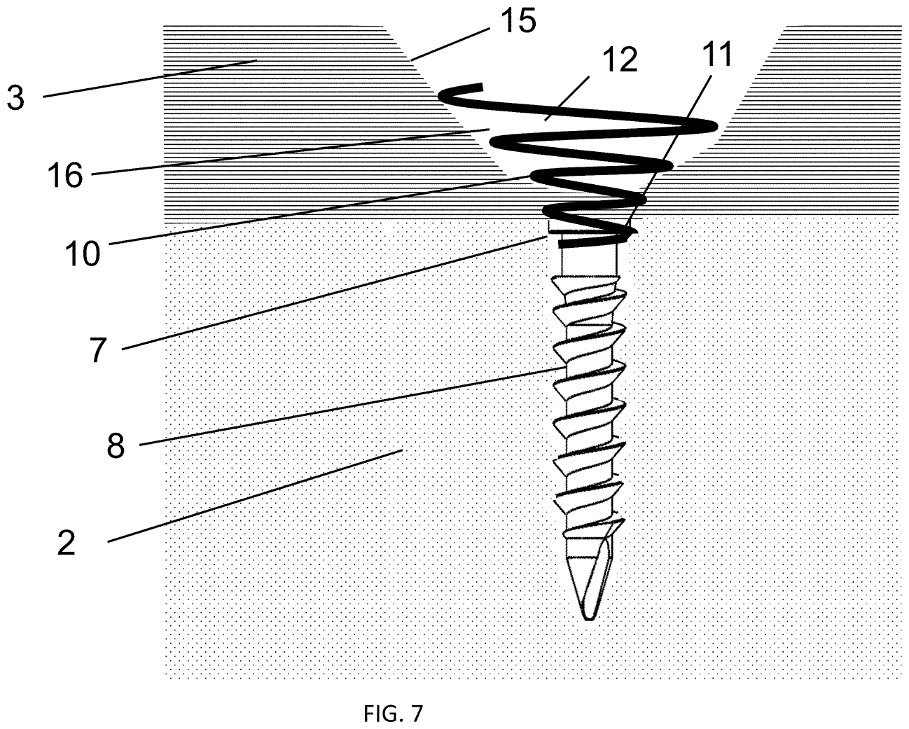

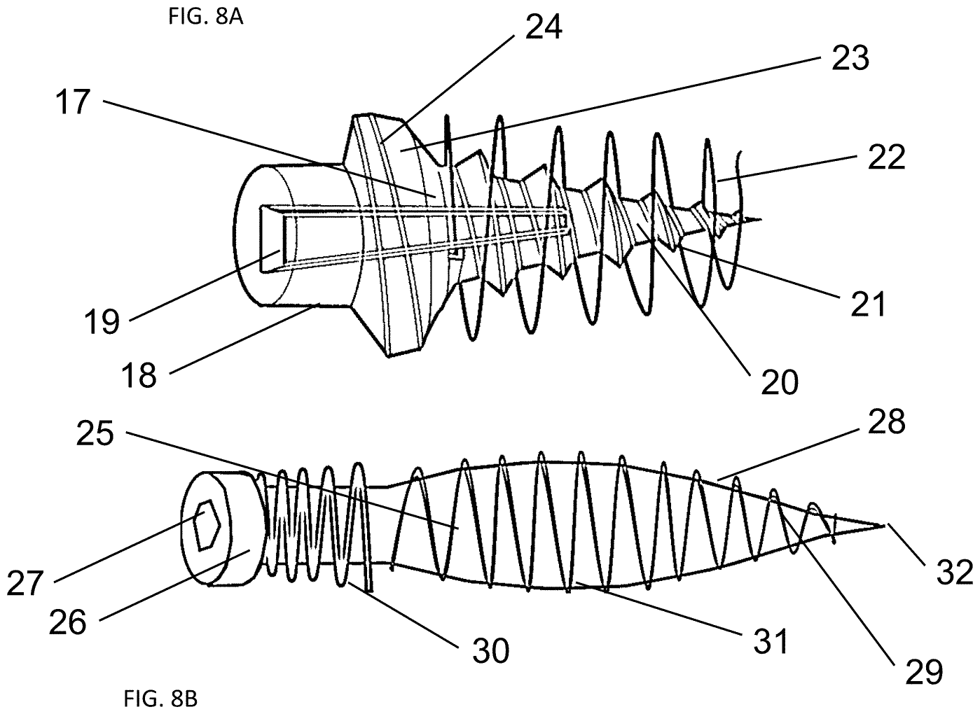

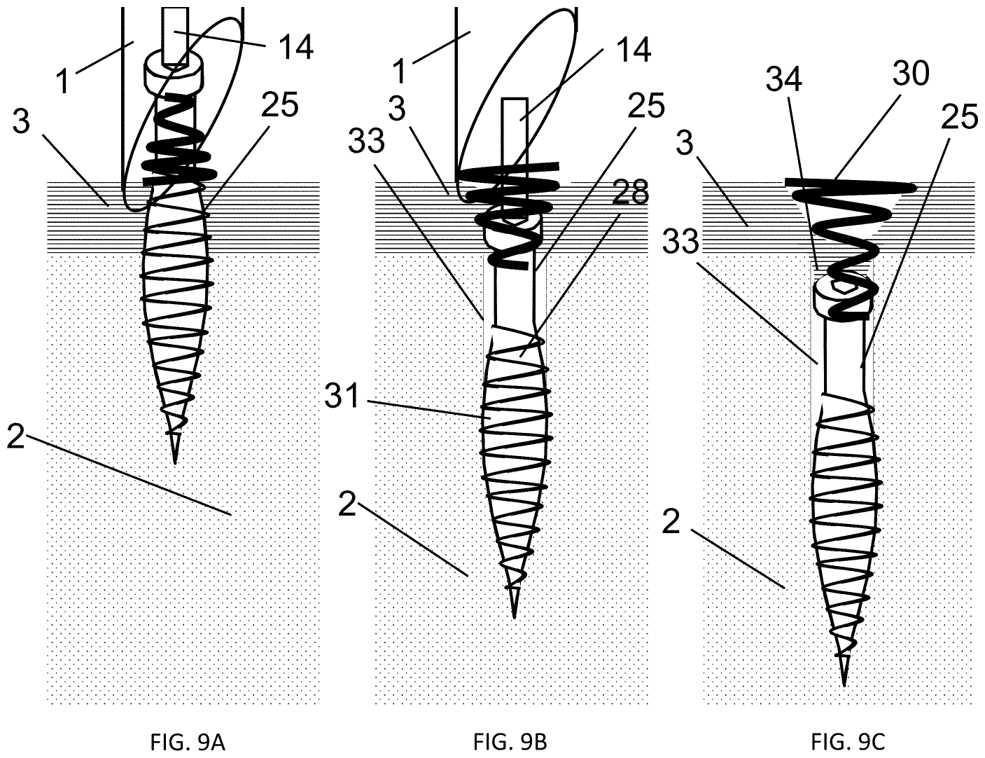

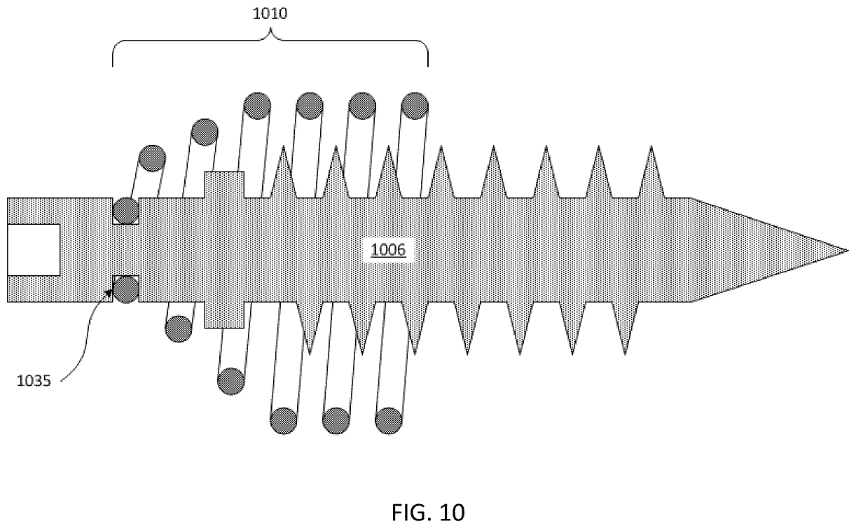

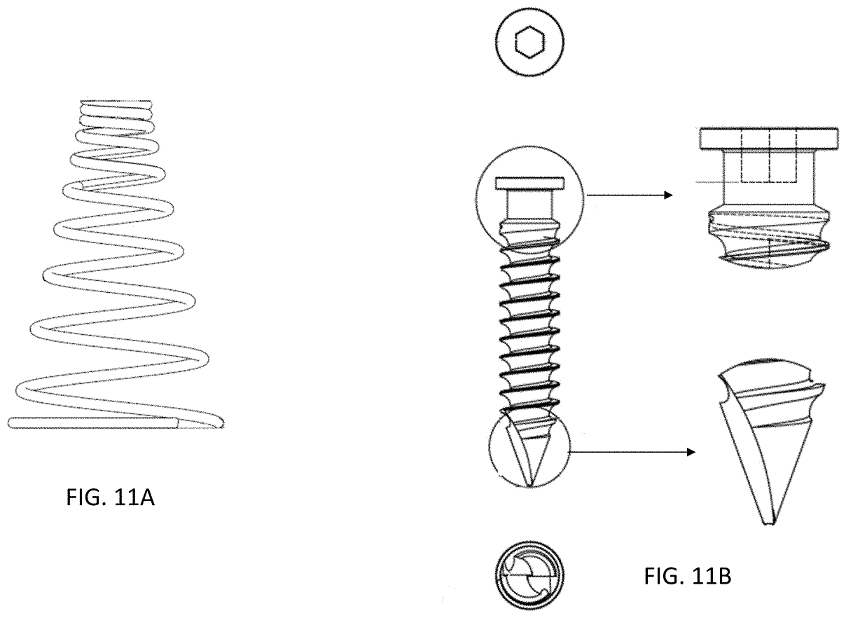

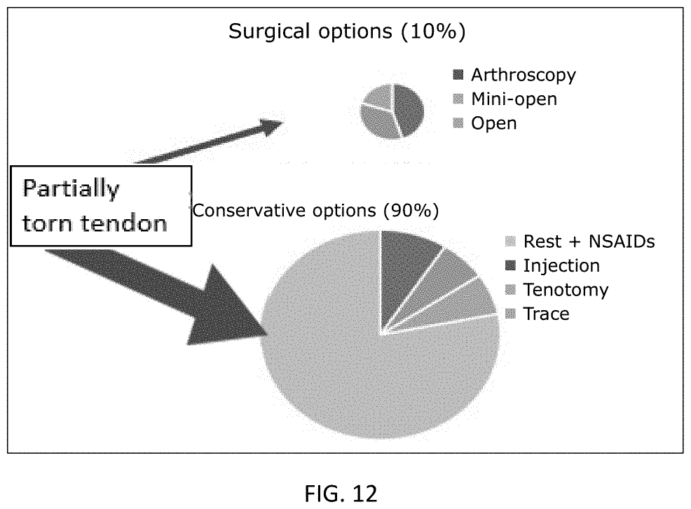

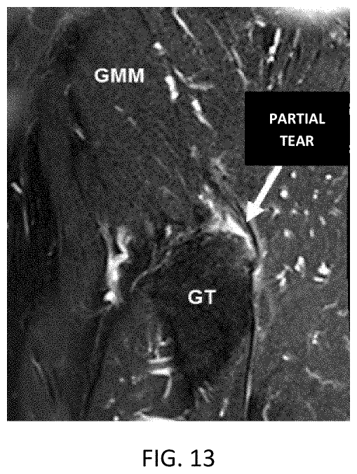

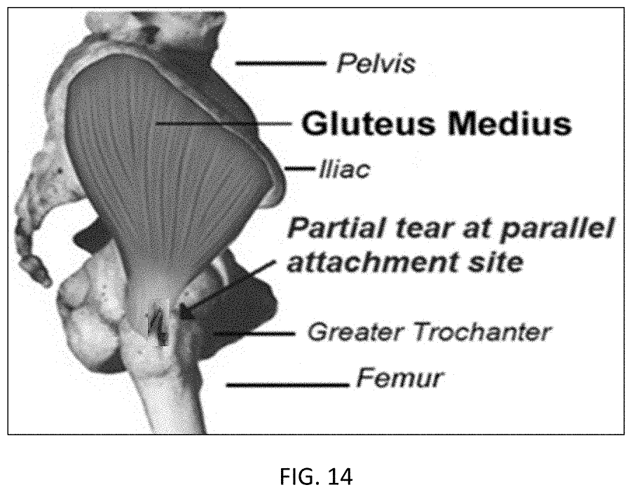

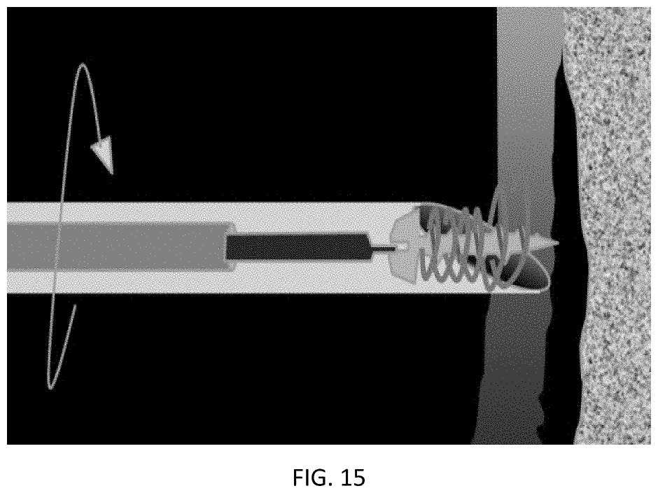

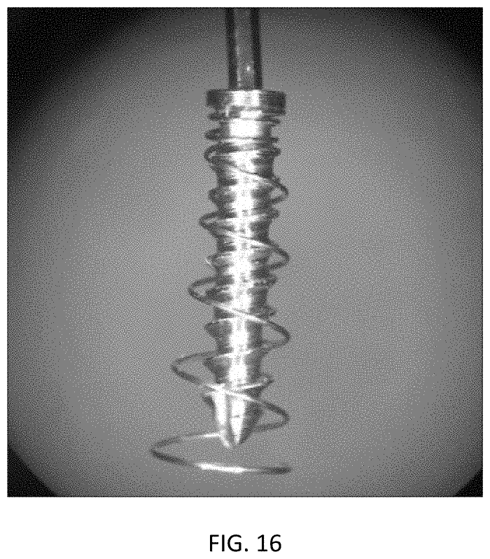

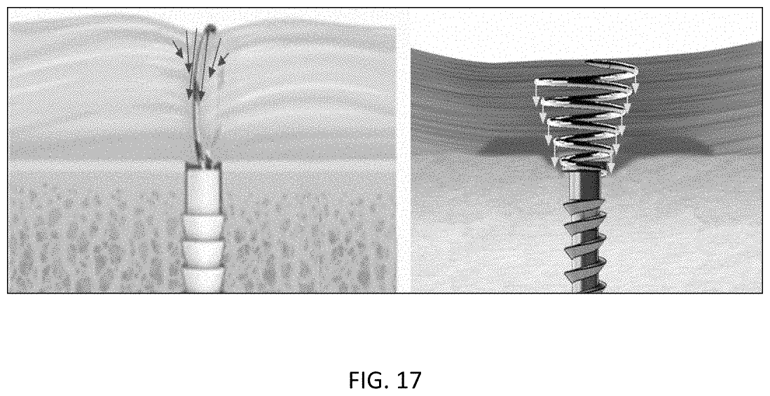

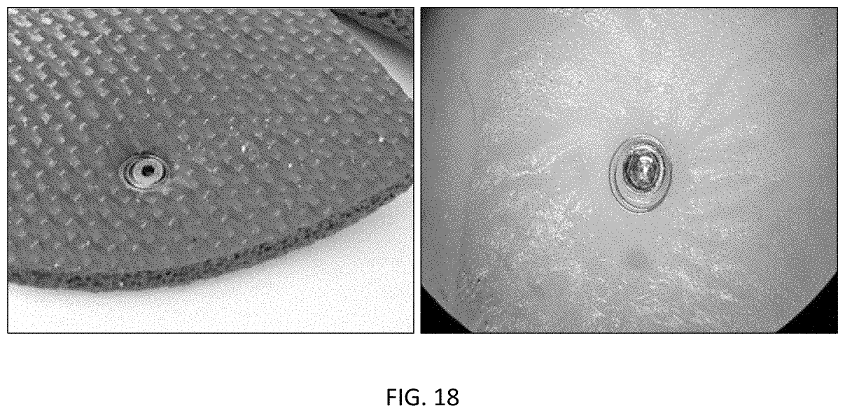

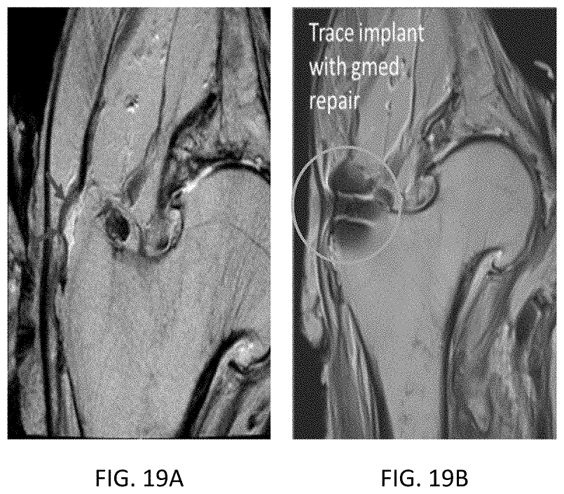

FIG. 6 depicts an advanced screw, with the coil deployed and inverted and the soft tissue compressed to the bone.

FIG. 7 depicts an advanced screw, with the coil deployed and inverted and the soft tissue compressed to the bone leaving a depressed area at the surface.

FIGS. 8A and 8B depict alternative screw designs. FIG. 8A depicts a screw with a collar of greater diameter relative to the screw hub and shaft and the coil wound from a position starting beneath the collar; FIG. 10B depicts a screw with a core region in the shaft of greater diameter relative to the rest of the shaft and the coil wound from a position beneath the screw head and above the widened core of the shaft.

FIGS. 9A-9C depict insertion of the alternative design screw of FIG. 8B. FIG. 9A depicts advancing the screw into the bone to secure the soft tissue to the bone; FIG. 9B depicts an advanced screw, with the coil deployed and inverted and the large screw shaft creating trailing chamber in bone; FIG. 9C depicts an advanced screw, with the coil deployed and inverted and the soft tissue compressed to the bone leaving a depressed area at the surface and tissue pulled into bone defect created by the large screw.

FIG. 10 depicts a headless screw design according to an embodiment of the invention.

FIG. 11A depicts an exemplary coil design according to an embodiment of the invention having a closed top end and bottom end. FIG. 11B depicts an exemplary spring washer screw as contemplated herein. A three-view drawing of the screw is shown in the left panel of FIG. 11B. The spring washer screw includes a symmetric self-tapping feature. The zoomed-in view of the head of the screw depicts the engagement of the spring washer with the screw. Embodiments of the spring washer screw also include a chisel tip.

FIG. 12 depicts a diagram illustrating the breakdown treatment options for partial tendon tears.

FIG. 13 depicts an MRI image of a hip showing a partial undersurface tear of the gluteus medius tendon (GMM) at the greater trochanter (GT).

FIG. 14 depicts an image of hip abductor anatomy, illustrating a gluteus medius partial tear.

FIG. 15 depicts a diagram of an exemplary percutaneous fastener placement at a lesion site between tendon and bone.

FIG. 16 depicts an image of an exemplary coil loaded onto a screw.

FIG. 17 depicts exemplary force profile diagrams for a standard suture anchor (left) and a tendon fastener according to embodiments of the present invention.

FIG. 18 depicts exemplary fastener prototypes according to embodiments of the present invention placed in synthetic models.

FIGS. 19A and 19B depict MRI images of partial gluteus medius tears on cadavers. FIG. 19A depicts a partial tear highlighted by arrows. FIG. 19B depicts a fasteners according to the present invention in a partial tear in a cadaver.

### DETAILED DESCRIPTION OF THE FIGURES

Soft tissue injuries can be particular debilitating to both young and old alike. Male and female athletes are commonly faced with debilitating adductor and abdominal tears, where the tendon or muscle is disengaged from the bone. To repair these injuries, platelet rich plasma has been utilized, with unproven results. Other options include open surgical procedures, but these face long recovery times.

For older patients (e.g., those over age of 65), age-related injuries include risk of hip fracture related to gluteus tendon tears. These common injuries result in weakness of the gluteus muscles and leads to sagging of the pelvis, tilting and to Trendelenburg gait. Ultimately, these weaknesses lead to debilitation and increase the risk of falls. Hip fractures occur at nearly 250,000 a year in the United States, and lead to significant impairment and risk of mortality.

The surgical procedures that are available are inadequate as they have significant recovery times and for certain patients increase the risk of secondary infection or other disease progression. Accordingly, new strategies are necessary to repair soft tissues that are disengaged from bone. Herein, embodiments describe an anchor system comprising a screw and a coil suitable for engaging soft tissue and bone.

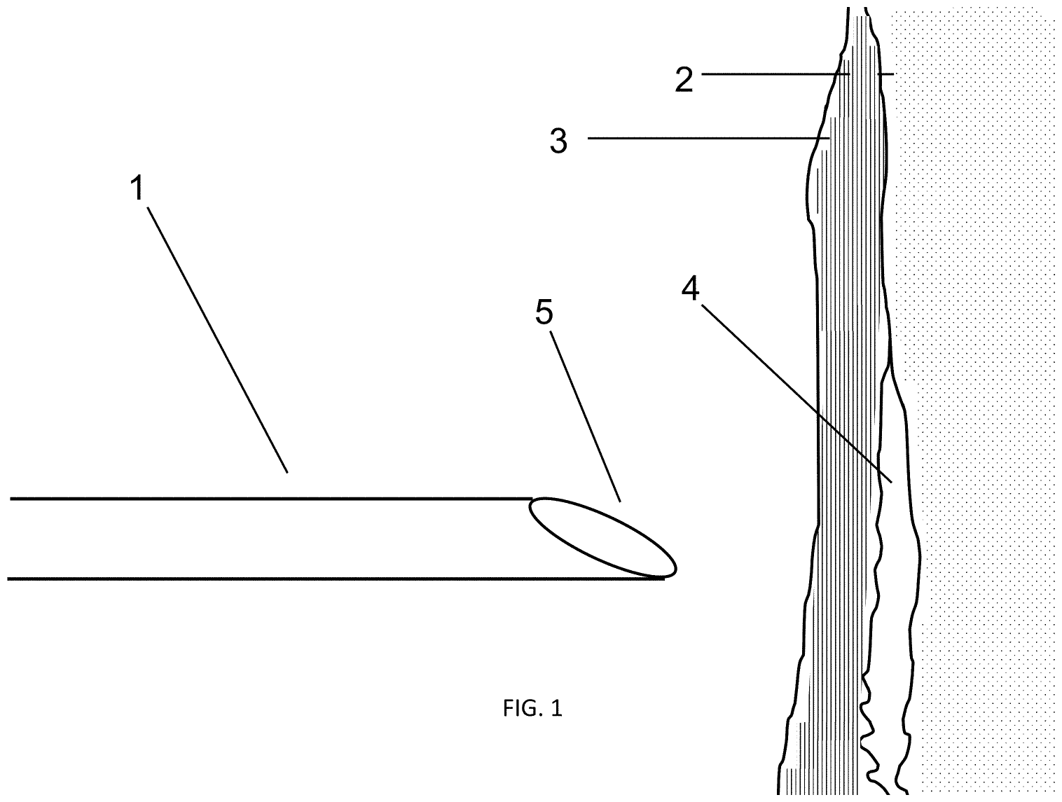

FIG. 1 depicts a soft tissue 3, generally a muscle or a tendon that is disengaged from the bone 2. The disengagement space 4 is the injury that is being rectified by the surgical tool described herein. In a healthy scenario, the soft tissue 3 is attached to the bone 2. The presence of disengagement space 4 is the injury, where the soft tissue 3 is separated from the bone 2.

FIG. 1 further depicts a needle entering the tissue space. The needle 1 is inserted into the tissue around the injury under normal insertion protocols. This can be completed with imaging guidance, by hand, or by other means as is known to a person of ordinary skill in the art. The needle aperture 5 is defined to allow for deployment of a tool for insertion of the device screw as depicted in further figures.

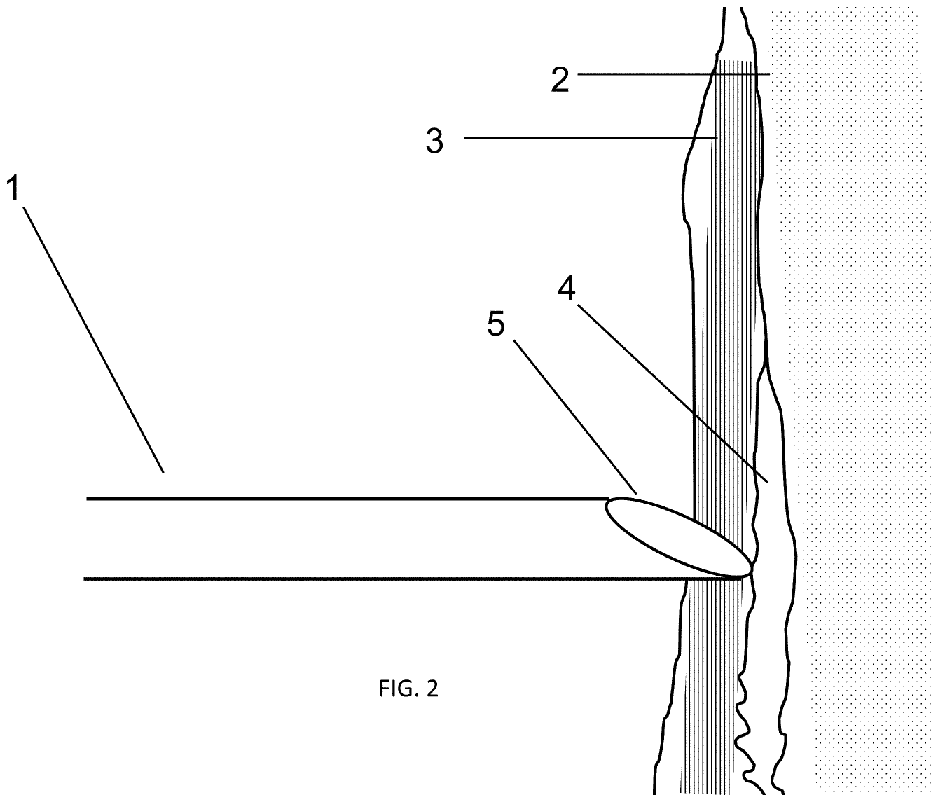

FIG. 2 depicts a further advancement of the needle 1 into the soft tissue 3, with the tip of the needle 1 inserted into the soft tissue 3. For deployment, the needle can also be adjacent to the soft tissue, allowing the tip of the screw or the coil to first penetrate the soft tissue, and allowing for deployment of the anchor as described herein.

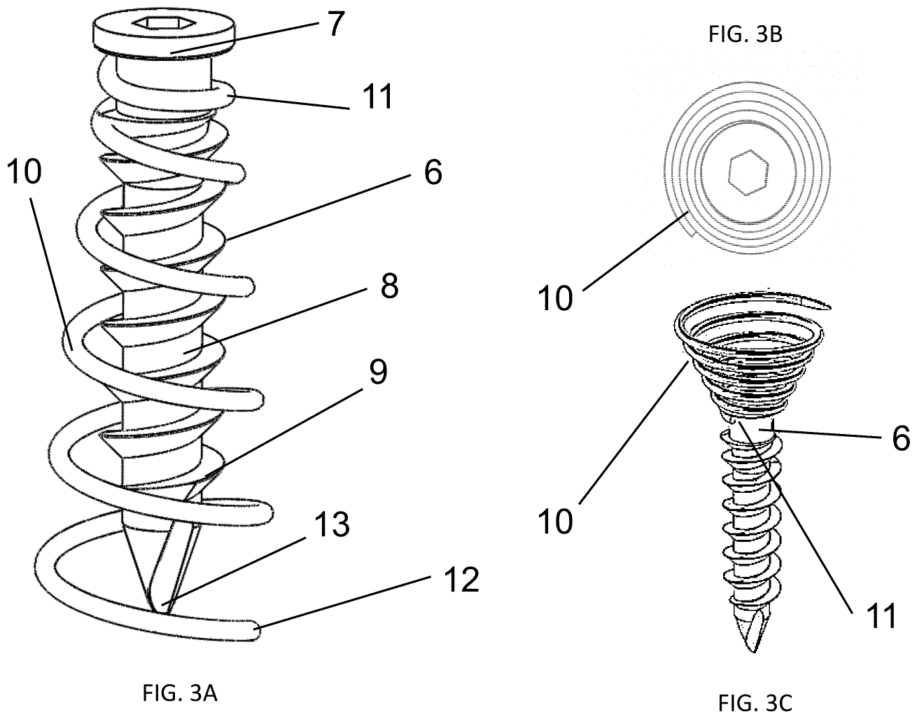

FIGS. 3A, 3B, and 3C depict an orthopedic anchor that is used to attach the soft tissue 3 to the bone 2 of FIGS. 1 and 2; FIG. 3A depicts a screw 6 with a coil 10 wound around it. Although a screw 6 is the most likely fastener to be utilized for surgery (e.g., in view of its holding power and ease of driving and removal), embodiments of the invention can be applied to other fasteners such as nails, pins, rivets, bolts, and the like.

The orthopedic anchor 6 comprises a head 7 and a shaft 8 with threads 9. The bottom face of head 7 engages with the coil 10 comprising a tapered end 11 and a flared end 12. The coil 12 is wound around shaft 8 with the tapered end positioned at the site of the bottom face of head 7 and the flared end positioned at the distal end of screw 6. In certain embodiments, the screw 6 may comprise a self-tapping region 13. In some embodiments, the screw is a self-drilling screw. That is, in some embodiments, the self-drilling screw does not require a pilot hole to be pre-drilled. FIG. 3B depicts a top view of the anchor of FIG. 3A looking down from above the top face. From this view, the coil 10 can appear as tightly packed concentric circles. FIG. 3C depicts the anchor of FIGS. 3A and 3B with coil 10 inverted (as it would be after driving through soft tissue 3 and into the bone 2. In this configuration, the tapered end 11 of coil 10 is engaged with the top portion of screw 6, directly contacting screw head 7 depicted in FIG. 3A.

The screws herein can have a variety of dimensions that can be selected to achieve desired biomechanical performance. For example, the screw can have a length between about 5 mm and about 15 mm, between about 8 mm and about 12 mm, about 10 mm, and the like. The coil 10 and/or the screw 6 may be manufactured out of a bio-absorbable material, so that after a pre-determined amount of time, each component can be absorbed into the body. However, it may also be suitable to have each component be manufactured of a non-bio-absorbable material, but simply a biocompatible material, for permanent positioning in the body. Alternatively, it may be advisable to have certain components be bio-absorbable and others nonabsorbable, for example, the coil may bio-absorb, but the screw may be non-absorbable. Any combination of absorbable or non-absorbable can be utilized as necessary.

The surgical screw 6 preferably possesses properties to allow for it to penetrate bone and anchor within the bone tissue. Accordingly, the screw 6 is preferably rigid to enable such penetration. Where the screw is maintained in the body, the screw is preferably poly(ether ether ketone) or another polymer, stainless steel (316L) or titanium, or another metal or alloy possessing similar flexural strength, pull-out strength, and stiffness. Exemplary Magnesium-based alloys include Mg—Ca—Sr, Mg—Zn, Mg-6Nz, Mg—Zn—Ca, MGYREZr, and the like. Iron-based alloys include Fe—Mn and the like.

In certain embodiments, the screw itself is bioresorbable and thus degrades in the body and is replaced by tissue ingrowth. However, to ensure that the mechanical properties of the screw are maintained for sufficient duration to enable healing of the injury, the mechanical properties can be maintained for at least 3 months, and preferably at least 6 months, before onset of degradation. Ultimately, complete degradation of an FDA-approved biomaterial and replacement of said material with tissue ingrown is desired. Suitable materials include, but are not limited to, certain polymers such as PLLA, PLDLA (e.g., 70:30, 80:20 L/L); PLGA (e.g., 50:50 L/L); PLLA-PLGA block copolymers; poly (para-dioxanone) (PPD); poly (propylene fumarate) (PPF) and the like.

In certain embodiments, the screw may be manufactured of a composite having a coating polymer or metal bulk material or ceramic particular-reinforced polymer matrix. In certain instances, a coating/filler material might include CaP, Tricalcium phosphate; Hydroxyapatite (HA), and similar materials known for biocompatibility and use within the human body.

The coil material can also have independent properties that assist in enabling the coil to effectively grip the soft tissue and to affix the soft tissue to adjacent bone tissue. In particular, the coil can possess shape memory, which limits its total deformation during deployment. This ensures that the coil is not simply deformed, but instead maintains sufficient rigidity and shape to grip into the soft tissue to enable the contact between tissue and bone for reattachment of the soft tissue.

The coil material may be manufactured of various polymers or metals, enabling both permanent and also bioresorbable production. The material preferably possesses physical properties similar to nitinol or platinum in flexural strength, pull-out strength, and stiffness. Where the coil is bioresorbable, like the screw above, the material can have mechanical properties maintained for at least 3 months, and preferably for at least 6 months to enable healing of the injury before the onset of degradation. However, the material can completely degrade and be replaced by tissue ingrowth after a set amount of time.

Suitable materials may include PLLA: PVA, PEG, PLA, poly(caprolactone) (PCL, ε-PCL), PLLA-PLGA, PEG-PCL, Chitosan (e.g., genipin-crosslinked) or other materials having similar profile for biocompatibility and bioresorbable. Where the material is maintained, metal alloys (e.g., spring steel), composites and combinations thereof, including the same materials as the screw (detailed above) are suitable.

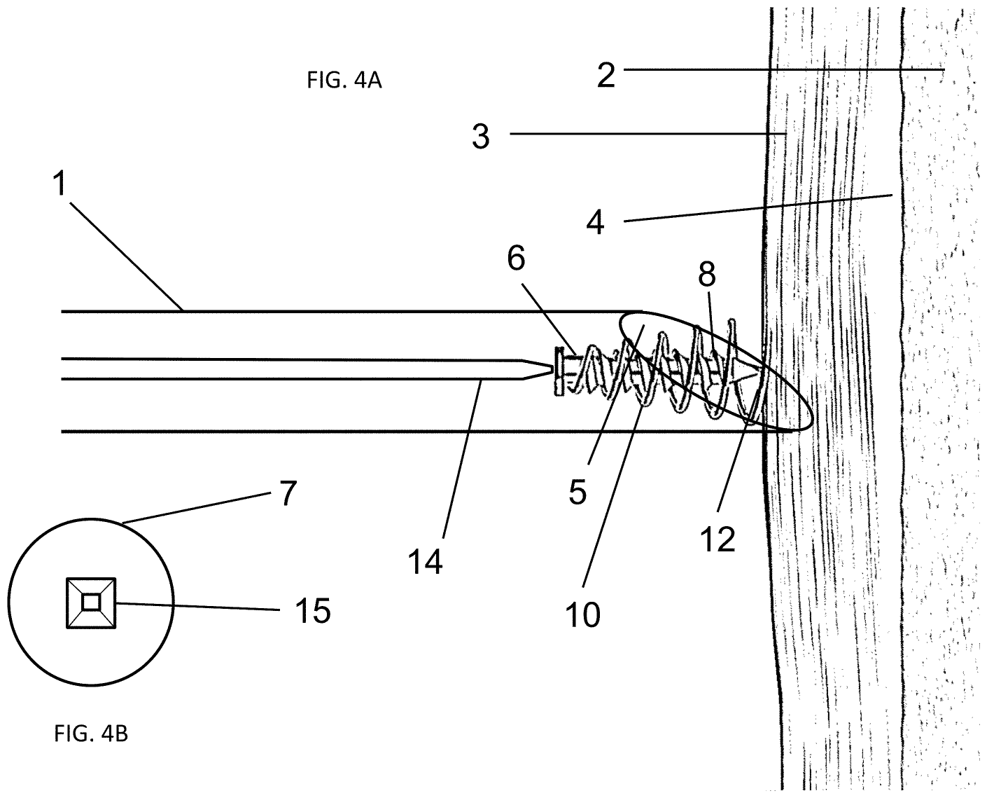

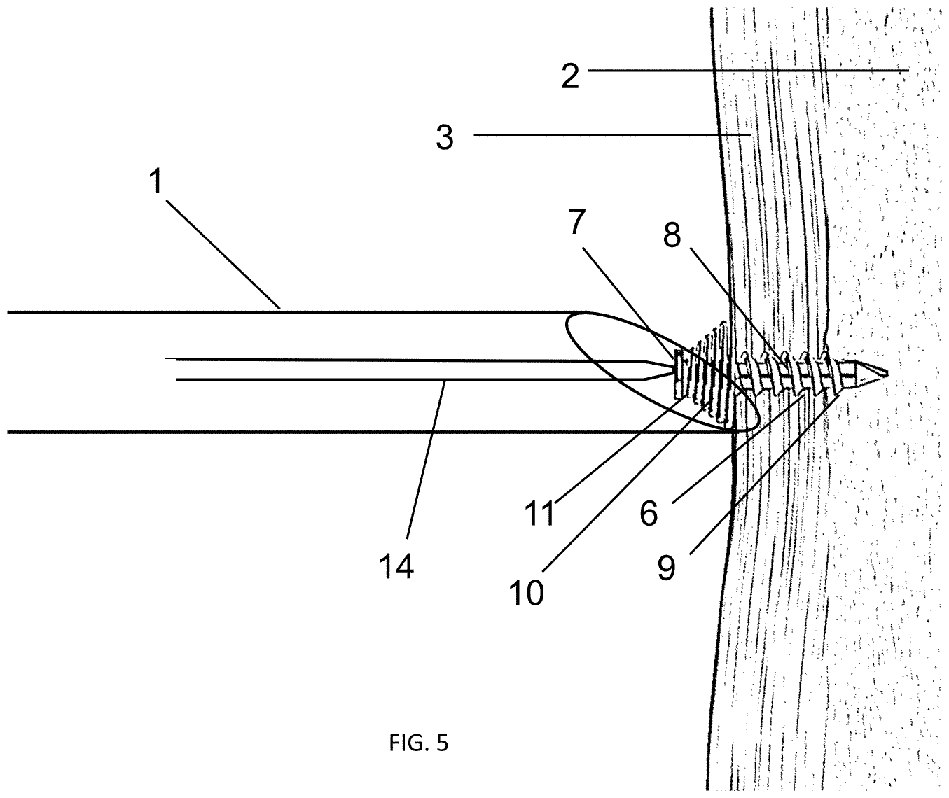

FIG. 4A depicts the anchor of FIGS. 3A and 3B positioned inside needle 1 engaged with a tool 14 to turn the screw 6. The screw 6 remains partially inside the needle aperture 5, with only a portion of the coil 10 and the shaft 8 projecting out of the aperture 5. The flared end of the coil 12 is pressed into the soft tissue 3; FIG. 4B depicts the top face of screw head 7 containing an inlet means 15 for turning screw 6. A pyramid-shaped inlet is shown and certain embodiments may include alternative shapes, for example hexagonal. Other suitable drives include slotted, cross, cruciform, Phillips, Frearson, French recess, JIS B 1012, Mortorq, Pozidriv PV, Supadriv PZ, Torq-set, Phillips/slotted, external polygon, square, pentagonal, hexagonal, 12-point, internal polygon, triangle, Robertson, hexagonal (Allen), double square, triple square, 12-spline flange, double hex, Torx, Torx Plus, Polydrive, three-pointed, tri-point, tri-groove, tri-wing, Bristol, Quadrex, Pentalobe, spanner (pig nose), and the like.

Screw 6 can have a variety of head geometries including a pan, button, dome, round, mushroom, truss, countersunk, flat, oval, raided, bugle, cheese, fillister, or flanged. In some embodiments, the head includes a flange that is substantially perpendicular to the shaft of the screw 6.

In some embodiments, the head diameter of the screw 6 is sized to retain coil 10, which can be wrapped around the shaft of the screw 6. In order facilitate inversion of the coil 10 during driving, the head diameter can be relatively small relative to the major diameter of the threads. For example, the screw head can have a diameter relative the major diameter of the threads between 110% and 100%, between 100% and 90%, and the like. The screw diameter (e.g., threads, shaft, and/or head) may include from about 1 mm to about 1.5 mm, from about 1.5 mm to about 2 mm, from about 2 mm to about 2.5 mm, from about 2.5 mm to about 3 mm, from about 3 mm to about 3.5 mm, from about 3.5 mm to about 4 mm, from about 4 mm to about 4.5 mm, from about 4.5 mm to about 5 mm, from about 5 mm to about 5.5 mm, from about 5.5 mm to about 6 mm, from about 6 mm to about 6.5 mm, from about 6.5 mm to about 7 mm, from about 7 mm to about 7.5 mm, from about 7.5 mm to about 8 mm, from about 8 mm to about 8.5 mm, from about 8.5 mm to about 9 mm, from about 9 mm to about 9.5 mm, from about 9.5 mm to about 10 mm and any and all increments therebetween.

In some embodiments, the screw 6 is a headless screw, in which case the coil 10 could engage with the screw 6 and rotate in the driven direction. Coil 10 can have a pitch opposite to the threads of the screw so that the distal end of the coil rotates over the surface of the soft tissue 3, but does not pierce the soft tissue 3. FIG. 10 depicts a headless screw 1006 including an annular recess 1035 adapted and configured to receive coil 1010. Screw 1006 can optionally rotate within the coil 1010. The coil may have a wire thickness of from about 0.05 mm to about 0.1 mm, from about 0.1 mm to about 0.15 mm, from about 0.15 mm to about 0.2 mm, from about 0.2 mm to about 0.25 mm, from about 0.25 mm to about 0.3 mm, from about 0.3 mm to about 0.35 mm, from about 0.35 mm to about 0.4 mm, from about 0.4 mm to about 0.45 mm, from about 0.45 mm to about 0.5 mm, from about 0.5 mm to about 0.55 mm, from about 0.55 mm to about 0.6 mm, from about 0.6 mm to about 0.65 mm, from about 0.65 mm to about 0.7 mm, from about 0.7 mm to about 0.75 mm, from about 0.75 mm to about 0.8 mm, from about 0.8 mm to about 0.85 mm, from about 0.85 mm to about 0.9 mm, from about 0.9 mm to about 0.95 mm, from about 0.95 mm to about 1 mm, and any and all increments therebetween. By another measure, in some embodiments, the wire has a gauge number in the range of from about 18 g to about 20 g, from about 20 g to about 22 g, from about 22 g to about 24 g, from about 24 g to about 26 g, from about 26 g to about 28 g, from about 28 g to about 30 g, from about 30 g to about 32 g, from about 32 g to about 34 g, from about 34 g to about 36 g, from about 36 g to about 28 g, from about 28 g to about 29 g, from about 29 g to about 30 g, from about 30 g to about 31 g, from about 31 g to about 32 g, from about 32 g to about 34 g, from about 34 g to about 36 g, from about 36 g to about 38 g, from about 38 g to about 40 g, from about 40 g to about 42 g, and any and all increments therebetween.

FIG. 5 depicts insertion of the anchor. When the screw 6 is turned by tool 14, the screw shaft 8 traverses soft tissue 3 and enters the bone 4 and flared end 12 of coil 10 presses into the soft tissue 3. This can be visualized by the coil 10 having fewer windings tightly wrapped around the shaft 8 and the outer windings spread wider into or over the soft tissue. The anchor, including the screw and coil, when the coil is wound around the screw, can have an outer diameter such that the anchor is sized to fit within, for example, a 10 g delivery cannula. In some embodiments, the anchor is sized to fit within an 8 g cannula, and 8 g to 10 g cannula, a 10 g to 12 g cannula, a 12 g to 14 g cannula, a 14 g to 16 g cannula, a 16 g to 18 g cannula, an 18 g to 20 g cannula, a 20 g to 22 g cannula, a 22 g to 24 g cannula, and any and all increments therebetween. The coil can deform inwardly and longitudinally when inserted into the needle before expanding radially as an end emerges from the distal end of the needle.

The coil can have shape memory to return to a previously formed shape after emerging from a distal end of the need. For example, the coil can have a resting length and diameter. For example, the coil can have a length between about 5 mm and about 15 mm, between about 8 mm and about 12 mm, about 10 mm, and the like. In some embodiments, the coil can have a length less than screw (e.g., terminating proximal of the distal end of the screw when engaged with the screw).

The coil diameter (e.g., outer diameter and the proximal or distal end) may be from about 1 mm to about 1.5 mm, from about 1.5 mm to about 2 mm, from about 2 mm to about 2.5 mm, from about 2.5 mm to about 3 mm, from about 3 mm to about 3.5 mm, from about 3.5 mm to about 4 mm, from about 4 mm to about 4.5 mm, from about 4.5 mm to about 5 mm, from about 5 mm to about 5.5 mm, from about 5.5 mm to about 6 mm, from about 6 mm to about 6.5 mm, from about 6.5 mm to about 7 mm, from about 7 mm to about 7.5 mm, from about 7.5 mm to about 8 mm, from about 8 mm to about 8.5 mm, from about 8.5 mm to about 9 mm, from about 9 mm to about 9.5 mm, from about 9.5 mm to about 10 mm and any and all increments therebetween. In some embodiments, the outer diameter of the distal end of the coil is 2.5 mm, which would fit within a 10G or 11G needle without deformation.

By turning screw 6 using tool 14, the threads of the screw shaft 9 engage with the bone 2 and at the same time, there is pressure on the tapered end 11 of coil 10 from the bottom face of screw head 7, causing coil 10 to compress during advancement of screw 6 into bone 2. As the screw 30 advances into the bone 2, the disengagement space 4 of FIGS. 1, 2 and 4A is removed. A comparison between FIGS. 4A and FIG. 5 shows the disengagement space 4 is removed.

FIG. 6 depicts further insertion of the anchor. As the screw 6 is further advanced into the bone 2 by tool 14, the flared end 12 of coil 10 maintains its position outside of the outer surface 15 of soft tissue 3. Meanwhile, the tapered end 11 of coil 10 maintains its position under the screw head causing the coil 10 to become inverted as shown in FIG. 3C. The soft tissue 3 is further pressed against the bone 2 creating an area of depression 16 surrounding coil 10. This allows the soft tissue 3 to heal and to re-attach to the bone 2 surface. It may be advantageous to add compounds or therapeutics to the surgical site to enhance the healing process. Furthermore, it may be advantageous to coat the various components, the coil, and/or screw, or washer with therapeutics to enhance the healing process.

FIG. 7 depicts the deployed anchor.

The screw shaft 8 is fully embedded into the bone 2. The coil 10 maintains the inverted orientation as the tapered end 11 is held underneath screw head 7 while the flared end 12 remained outside the surface 15 of soft tissue 3. An area of depression 16 is formed in the shape of a funnel by fastening the screw head 7 against the soft tissue 3, compressing the soft tissue 3 against the bone 2 as the site of insertion. The contact between the two tissues allows healing process to occur, where the soft tissue 3 is normally attached to the bone 2.

FIGS. 8A and 8B depict further embodiments of an orthopedic anchor. FIG. 8A depicts screw 17, which like FIG. 3A, comprises a head 18 comprising an inlet 19, a shaft 20 with threads 21, and a coil 22. However, this embodiment comprises a collar 23 with a greater cross-sectional area than that of shaft 20 and head 18. The collar 23 comprises threads 24 oriented in the same direction as threads 21 of shaft 20; FIG. 8B depicts screw 25, which like FIG. 3A, comprises a head 26 comprising an inlet 27, a shaft 28 with threads 29, and a coil 30. However, in this embodiment, shaft 28 comprises a section with a greater cross-sectional area 31 than the rest of shaft 28 and head 26 such that the shaft tapers from this section 31in both directions, toward the head 26 and screw tip 32, to form a candle-shape for shaft 28. Coil 30 is positioned between screw head 26 and the section with a greater cross-sectional area 31.

FIGS. 9A, 9B, and 9C depict deployment of the anchor comprising screw 25 depicted in FIG. 8B. FIG. 9A depicts insertion of the anchor comprising screw 25 into soft tissue 3 and bone 2 using needle 1 and tool 14 as depicted in FIG. 5 for the anchor comprising screw 6; FIG. 9B depicts further insertion of the anchor comprising screw 25 into soft tissue 3 and bone 2 using needle 1 and tool 14 as depicted in FIG. 6 for the anchor comprising screw 6. As the screw 25 is further advanced into the bone 2, an area of bone displacement 33 is formed in bone 2 as a result of the widened area 31 of screw shaft 28. FIG. 9C depicts the deployed anchor comprising screw 25 as depicted for the anchor in FIG. 7. However, in this case, a portion of soft tissue 34 surrounding coil 30 is pulled into the displacement area 33. This further allows the soft tissue 3 to heal and to re-attach to the bone 2 surface. It may be advantageous to add in certain compounds or therapeutics to the surgical site to enhance the healing process. Furthermore, it may be advantageous to coat the various components, the coil, and/or screw, or washer with certain therapeutics to enhance the healing process.

Certain bone or vascular stimulators can be utilized to stimulate healing at the surgical site. Indeed, these can be coated onto the screw and or coil, or combinations thereof, or injected to, or applied to the surgical area through the needle during the surgical procedure.

Further embodiments describe use of a dual-function anchor system to engage a displaced soft tissue, engage said soft tissue with a coil, and drive the soft tissue, having the embedded coil, to contact a bone, by driving a screw into the bone. Said dual-function anchor system comprises a surgical screw comprising a shaft having threads, and a coil; said coil wound around the screw shaft; said coil comprising a coil end which is defined to engage the bottom face of the screw head of said screw; engaging the end of the shaft and the coil to a soft tissue.

FIG. 11A depicts an additional embodiment of the coil, having one end that engages with the screw of the anchor system and the other end has a closed configuration. A “closed” configuration can include, but is not limited to, configurations in which the end of the coil is aligned distally under an adjacent helical curve such that the end contacts the adjacent helical curve, with or without fastening. In some embodiments, the end of the coil has a tapered edge so that when the end contacts the adjacent helical curve, the end is flush with the surface of the adjacent helical curve. When said anchor is advanced, the screw engages with bone while the closed end of the coil does not engage with soft tissue. In some embodiments, the coil according to this embodiment performs as a spring washer compressing as the screw is driven into the bone.

In certain preferred embodiments, and methods of treatment, it may be suitable to drive the dual-function anchor system comprising a screw and a coil into the tissue of a patient. Before or after engaging the tissues, the aperture for the dual-function anchor system may further be utilized to inject certain bone or vascular healing compositions to the surgical site.

Accordingly, methods of treatment may comprise the above methods, further comprising a step of injecting a therapeutic to the wound site, for example a bone or vascular healing composition. A further therapeutic may include antibiotics. In certain embodiments, the coil or the screw may be coated with a therapeutic, including a bone or vascular healing composition or an antibiotic. Those of ordinary skill in the art will recognize these compounds and their appropriate doses for administration. For example a non-limiting list of antibiotics may include clindamycin, trimethoprim/sulfamethoxazole, doxycycline, vancomycin, linezolid, daptomycin, metronidazole or combinations thereof.

### EXPERIMENTAL EXAMPLES

### Example 1

Musculoskeletal (MSK) injuries affect 20% of the general population and up to 55% of the population over the age of 60 years. Partial tendon tear is an extremely common type of musculoskeletal injury, and we estimate over 31 million in the US each year as countless cases go unreported. Lesions form as attachments to bone become damaged, causing pain and noticeable reduction in strength. The severity of the tear determines the recommended treatment, as surgery is only indicated for tears exceeding half the thickness. This leaves nearly 90% of the massive general patient population presenting with tendon tears underserved in the current paradigm as conservative treatment is heavily favored over surgical intervention. This is illustrated in the treatment approach breakdown displayed (FIG. 12), with corresponding options for each approach shown as well. Under conservative management, none of the existing activities has been proven to restore full functionality any faster than the natural healing process, including injectable bioactive agents. Surgical intervention normally yields better outcomes but is costly, time intensive, and has higher risk for complications. Doctors are reluctant to operate on partial tears, particularly when treating older patients. This shows a major gap in the current paradigm, with no intermediate procedure to deliver the benefit of surgery using a minimally invasive approach.

The significance of this unmet need is illustrated by the prevalence of partial tendon tears around the body. This is measured to be approximately 31 million in the US, with a serviceable market comprising partial tears having clean edges and no retraction or shredding of the tendon. This equals approximately 3 million cases involving rotator cuff, gluteus medius, hip adductor, epicondyle, and patella. These are candidates for a new mode of minimally invasive repair. For example, there has been recent interest among orthopedic surgeons to repair the gluteus medius tendon, thereby reducing the gait deficiency. Gait disturbance related to gluteus medius tendon tear and muscle atrophy has been identified as a significant cause of older patient falls. However, older patients are often poor surgical candidates due to co-morbid conditions (i.e. heart, lung or renal disease) making general anesthesia risky in this population and leaving the majority of cases untreated. In a study of 185 randomly selected MRI exams of the pelvis divided into 10-year age groups, partial tear of the gluteus medius tendon was observed in 31.8% in the 50-59 year category, 46.3% in the 60-69 age group, and in 61.7% of patients age 70 and above. An inexpensive, easily available non-surgical method for gluteus tendon repair could have a major impact on quality of life for older patients and reduce risk of falling and associated injuries.

### Tendon Reattachment

A solution is presented herein that can reattach the partially torn gluteus medius tendoneous insertion into the greater trochanter percutaneously, without a surgical procedure that would require general anesthesia. This technique involves placing implants into the tendon insertion site (footprint) through the partial tear. The implant is composed of two functional parts: a self-drilling screw and a tissue-capture coil. The coil is tightly bound to the neck of the screw adjacent to the hub. The coil windings have increasing circumference along its course, in a conical configuration. The screw and coil construct fits within a 10 gauge delivery cannula. The partial thickness tendon tear is identified by imaging, either MRI (FIG. 13) or ultrasound. Testing can be performed for patient selection, including steroid injection into the bursa adjacent to the tendon; if pain temporarily resolves it suggests that repair of the tendon has a higher likelihood of reducing the patient's symptoms. One important characteristic of amenable partial thickness tears is that the tendon remains at its normal location along its origin footprint at the iliac crest of the femur at the greater trochanter (FIG. 14). In an initial study using de-identified patient data, it has been determined that 100% of tears are well-defined at initial presentation with hip pain (i.e., not shredded) and all subsequently progress to full thickness tears followed by muscle atrophy within 2-4 years. Tissue bulk loss and healing deficiency caused by atrophy impede both mechanical fixation and biological repair. The reinforcement technique of the present invention for the partially torn tendon will arrest full tear progression and muscle atrophy, with patients in pain and with no atrophy early in their disease course as treatment candidates.

Using ultrasound guidance for the procedure, a common interventional radiology appliance that allows real-time visualization of implant placement, the tear is identified; the needle course is planned and the skin is marked. An insertion tool is envisioned (FIG. 15) in which a delivery cannula loaded with the implant is positioned at the site of tear. The system is passed percutaneously to the bone target using an integrated stylet applicator compatible with the screw hub. The outer needle sheath is retracted, exposing the device for implantation. The core is then advanced and turned clockwise, causing the anchor to enter the bone. Multiple implants can be placed for added reinforcement at the insertion site. The implant is rotated clockwise and advanced into the bone. As the screw enters the bone, the coil captures the overlying tendon, pulling it against and into the bone along with the screw. The coil acts as an expandable washer, like a funnel clasping the tendon surface.

### Attachment Techniques

The fastener according to the present invention is based on establishing the ability to effectively tack down partial thickness tears—particularly those painful partial tears that are not yet surgical candidates, but are not responding to conservative management techniques. While such a repair may not be as strong as a standard suture anchor, it is likely superior to existing needle-based procedures for partial thickness tears. Since the proposed technique achieves functional repair, this falls into the realm of surgery for medical reimbursement purposes, yet can be performed in a doctor's office or an imaging suite. By utilizing the interior space of a percutaneous needle to accommodate a device capable of achieving tissue fixation mechanically, our concept represents an innovative approach for treatment of painful partial tendon tear. The tendon fastener implant design of the present invention comprises a bone screw with standard clockwise threads with an attached conical compression spring as pictured (FIG. 16).

This changes the force profile with respect to the fixation as compared to a traditional suture anchor as diagrammed (FIG. 17). A suture anchor serves to pin the tendon against bone and there is a concentration of forces (arrows) along the single plane of the suture. There is also a single point of contact, the junction of the suture and the anchor eyelet. Together these create weakness in the implant, which is exacerbated by techniques introducing knots along the suture. Conversely, the fastener of the present invention enjoys a distribution of forces (arrows) along a wider area as a result of its multiple contact points within the tendon tissue, and it there is no eyelet to serve as a failure point. With the tendon also being pulled into the bone, the fastener of the present invention offers increased fixation strength and failure resistance.

### Results and Conclusion

In benchtop testing using synthetic bone and tendon tissue models, it was observed that as the anchor is inserted, the coil becomes slightly inverted over the screw head, creating a funnel and a whirlpool-like effect (FIG. 18). Data was collected using human hip cadavers. The gluteus medius tendon was examined in each. One was a clean partial tear, clearly seen on MRI image capture (FIG. 19A), and was selected for use as a model case. Labeled wooden skewers were placed into specimen at greater trochanter to localize insertion. An MRI was repeated and showed which skewer best localized the gluteus medius insertion site. The surface was marked with permanent marker, and a cutdown was performed along the skewer. Tensor fasciae latae was incised at the iliac crest. A vertical incision was reflected to each side, exposing the greater trochanter and gluteus medius insertion. The target site was implanted with an exemplary percutaneous fastener of the present invention using retractor and hex driver. Fixation is shown on MRI (FIG. 19B) with tendon funneled into the bony insertion site at the greater trochanter. Two other cadavers were partially torn and used in a comparative study with the self-drill device described herein and an Arthrex pre-drill suture anchor. Pullout failure was measured in the same direction as force would normally be exerted on the hip using a tensile meter. Failure testing was performed on the specimens by tensioning the repair construct longitudinally. A tensile meter (max 200 N, 0.01 N graduation) with setting on peak force measured was affixed to a platform. A metal wire was sewn into muscle above the tendon insertion in the specimens, and connected to the tensile meter via hook. Increasing manual tension was applied parallel to the tendon orientation until failure. The peak values at the point of failure were captured and compared for the cadaver specimens. The fastener of the present invention demonstrated a failure strength measured at 155.0 N, which 92.5% of that achieved with the suture anchor, measured at 167.6 N. This demonstrates that the fastener of the present invention can be used to successfully achieve tendon fixation.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference

1. A surgical anchor comprising:
a fastener; and
an external invertible coil coupled to a proximal end of the fastener.

2. The surgical anchor of claim 1, wherein the fastener is a screw comprising:
distal threads; and
a reamer proximal to the distal threads,
wherein the screw is a headless screw; and
the screw comprises an annular recess proximal to the reamer, the recess adapted and configured to receive a portion of the external invertible coil.

3. The surgical anchor of claim 2, wherein the screw comprises a head having an outer diameter equal to or less than an outer diameter of the reamer.

4. A method of repairing a soft tissue when disassociated from a bone, the method comprising:
driving the surgical anchor of claim 1 through the soft tissue and into the bone until at least a portion of the coil lies against the soft tissue proximal to the head of the screw.

5. A surgical tool comprising:
the surgical anchor of claim 1;
a needle comprising an aperture of sufficient diameter for receiving the surgical anchor; and
a tool adapted and configured for:
insertion into said aperture to insert the surgical anchor; and
engaging with and turning said surgical anchor.

6. A surgical anchor comprising:
a screw comprising:
distal threads; and
a proximal head; and
a coil coupled to the screw proximal to the distal threads;
wherein:
the screw can rotate freely within the coil in either rotational direction without driving the coil; or
the diameter of the coil expands distally.

7. A method of repairing a soft tissue when disassociated from a bone, the method comprising:
driving the surgical anchor of claim 6 through the soft tissue and into the bone until at least a portion of the coil lies against the soft tissue proximal to the head of the screw.

8. A surgical tool comprising:
the surgical anchor of claim 7;
a needle comprising an aperture of sufficient diameter for receiving the surgical anchor; and
a tool adapted and configured for:
insertion into said aperture to insert the surgical anchor; and
engaging with and turning said surgical anchor.

9. A surgical anchor comprising a screw and a coil; said coil having a conical shape, wound around the screw shaft with the first end of the coil being tapered and having a ring circumference smaller than that of the screw head and being engaged to the bottom of the screw head and the second end of the coil being flared with a larger ring circumference and positioned along the screw shaft.

10. A surgical system comprising a needle comprising an aperture of sufficient diameter for receiving a surgical anchor comprising a screw and a coil; a stylet tool, insertable into said aperture for inserting said anchor and capable of engaging with and turning the screw; said screw comprising a shaft having threads and a head, said head having a bottom face, and said coil having a conical shape, wound around the screw shaft with the first end of the coil being tapered and having a ring circumference smaller than that of the screw head and being engaged to the bottom of the screw head and the second end of the coil being flared with a larger ring circumference and positioned along the screw shaft.

11. A method of repairing a soft tissue when disassociated from a bone comprising: inserting a surgical anchor into a soft tissue which has become detached from bone, said system comprising an aperture for inserting surgical anchor comprising a screw and a coil, and a stylet tool insertable into said aperture for inserting said anchor and capable of engaging with and turning the screw to cause its entry into the bone; said screw comprising a shaft having threads, and a head, said head having a bottom face, and a coil; said coil having a conical shape, wound around the screw shaft with the first end of the coil being tapered and having a ring circumference smaller than that of the screw head and being engaged to the bottom of the screw head and the second end of the coil being flared with a larger ring circumference and positioned along the screw shaft.

12. A surgical anchor comprising a screw and a coil, said screw comprising a head, a shaft having threads, and a collar, said collar having a greater cross-sectional area than that of the shaft and the head.
